(19) United States
(12) Patent Application Publication
(10) Pub. No.: US 2005/0049691 A1
(43) Pub. Date: Mar. 3, 2005

(54) Polymeric reconstrainable, repositionable, detachable, percutaneous endovascular stentgraft

(76) Inventors: Robert A. Mericle, Gainesville, FL (US); Swadeshmukul Santra, Gainesville, FL (US); Matthew V. Burry, Gainesville, FL (US); Christopher D. Batich, Gainesville, FL (US); Courtney S. Watkins, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/652,557

(22) Filed: Sep. 2, 2003

(51) Int. Cl. A61F 2/06

(52) U.S. Cl. 623/1.23

(57) ABSTRACT

A coaxial double lumen tube adapted for forming an endovascular graft which comprises an outer tube positioned over an inner tube, both tubes being made of a material acceptable for use in endovascular grafts and having an internal and external diameter and a wall thickness, the outer tube having an internal diameter and the inner tube having an external diameter such that a space is created between the outer tube and inner tube, said space being at least partially filled with an uncured adhesive which, upon curing after endovascular implantation, cures to adhere said inner and outer tubes together to form a self-supportive tube.

### BACKGROUND OF THE INVENTION

1. Field of the Invention

The invention relates to a system and method for the treatment of the vasculature.

2. Description of the Prior Art

The present invention relates to a system and method for the treatment of disorders of the vasculature. Such conditions require intervention due to the severity of the sequelae, which frequently is death. Prior methods of treating aneurysms and similar defects in the vasculature include invasive surgical methods with graft placement within the affected vessel as a reinforcing member thereof. However, such procedures require a surgical cut down to access the vessel, which in turn can result in a catastrophic rupture of the defect due to the decreased external pressure from the surrounding organs and tissues, which are moved during the procedure to gain access to the vessel. Accordingly, surgical procedures have a high mortality rate due to the possibility of the rupture discussed above in addition to other factors. Such other factors can include poor physical condition of the patient due to blood loss, anuria, and low blood pressure associated with the aortic abdominal aneurysm. An example of a typical surgical procedure is that described in a book entitled Surgical Treatment of Aortic Aneurysms by Denton A. Cooley, M. D., published in 1986 by W. B. Saunders Company.

Due to the inherent risks and complexities of surgical procedures, various attempts have been made in the development of alternative methods for deployment of grafts within blood vessels. One such method is the non-invasive technique of percutaneous delivery by a catheter-based system. Such a method is described in Lawrence, Jr. et al in “Percutaneous Endovascular Graft: Experimental Evaluation”, Radiology (May 1987). Lawrence described therein the use of a Gianturco stent as disclosed in U.S. Pat. No. 4,580,568. The stent is used to position a Dacron.RTM. fabric graft within the vessel. The Dacron.RTM. graft is compressed within the catheter and then deployed within the vessel to be treated. A similar procedure has also been described by Mirich et al. in “Percutaneously Placed Endovascular Grafts for Aortic Aneurysms: Feasibility Study”, Radiology (March 1989). Mirich describes therein a self-expanding metallic structure covered by a nylon fabric, with said structure being anchored by barbs at the proximal and distal ends.

One of the primary deficiencies of the existing percutaneous devices and methods has been that the grafts and the delivery catheters used to deliver the grafts are relatively large in profile, often up to 24 French and greater, and stiff in bending. The large profile and bending stiffness makes delivery through the irregular and tortuous arteries of diseased vessels difficult and risky. In particular, the iliac arteries are often too narrow or irregular for the passage of a percutaneous device. In addition, current devices are particularly challenged to reach the deployment sizes and diameters required for treatment of lesions in the aorta and aorta-iliac regions. Because of this, non-invasive percutaneous graft delivery for treatment of aortic aneurysm is not available to many patients who would otherwise benefit from it.

Elastomeric vascular grafts are also known. They are manufactured, for example, by methods which incorporate electrostatic spinning technology such as that described by Annis et al. in “An Elastomeric Vascular Prosthesis”, Trans. Am. Soc. Artif. Intern. Organs, Vol. XXIV, pages 209-214 (1978) and in U.S. Pat. No. 4,323,525. Other approaches include elution of particulate material from tubular sheeting, such as by incorporating salts, sugars, proteins, water-soluble hydrogels, such as polyvinyl pyrrolidone, polyvinyl alcohol, and the like, within polymers and then eluting the particulate materials by immersion in water or other solvent, thereby forming pores within the polymer. Exemplary in this regard is U.S. Pat. No. 4,459,252. Another approach involves the forming of pores in polymers by phase inversion techniques wherein a solventized polymer is immersed in another solvent and the polymer coagulates while the polymer solvent is removed. Also known are spinning techniques such as those described in U.S. Pat. No. 4,475,972. By that approach, a polymer such as a polyurethane in solution is extruded as fibers from a spinnerette onto a rotating mandrel. The spinnerette system reciprocates along a path which is generally parallel to the longitudinal axis of the mandrel and at a controlled pitch angle. The result is a non-woven structure where each fiber layer is bound to the underlying fiber layer.

Also known are stent devices, which are placed or implanted within a blood vessel or other body cavity or vessel for treating occlusions, stenoses, aneurysms, disease, damage or the like within the vessel. These stents are implanted within the vascular system or other system or body vessel to reinforce collapsing, partially occluded, weakened, diseased, damaged or abnormally dilated sections of the vessel. At times, stents are used to treat disease at or near a branch, bifurcation and/or anastomosis. This runs the risk of compromising the degree of patency of the primary vessel and/or its branches or bifurcation, which may occur as a result of several problems such as displacing diseased tissue, vessel spasm, dissection with or without intimal flaps, thrombosis and embolism.

One common procedure for implanting a stent is to first open the region of the vessel with a balloon catheter and then place the stent in a position that bridges the diseased portion of the vessel. Various constructions and designs of stents are known. U.S. Pat. No. 4,140,126 describes a technique for positioning an elongated cylindrical stent at a region of an aneurysm to avoid catastrophic failure of the blood vessel wall, the stent being a cylinder that expands to an implanted configuration after insertion with the aid of a catheter. Other such devices are illustrated in U.S. Pat. No. 4,787,899 and U.S. Pat. No. 5,104,399. U.S. Pat. No. 4,503,569 and U.S. Pat. No. 4,512,338 show spring stents which expand to an implanted configuration with a change in temperature. It is implanted in a coiled configuration and then heated in place to cause the material of the spring to expand. Spring-into-place stents are shown in U.S. Pat. No. 4,580,568. U.S. Pat. No. 4,733,665 shows a number of stent configurations for implantation with the aid of a balloon catheter. U.S. Pat. No. 5,019,090 shows a generally cylindrical stent formed from a wire that is bent into a series of tight turns and then spirally wound about a cylindrical mandrel to form the stent. When radially outwardly directed forces are applied to the stent, such as by the balloon of an angioplasty catheter, the sharp bends open up and the stent diameter enlarges. U.S. Pat. No. 4,994,071 describes a bifurcating stent having a plurality of wire loops that are interconnected by an elongated wire backbone and/or by wire connections and half hitches. Stents themselves often lead to undisciplined development of cells in the stent mesh, with rapid development of cellular hyperplasia. Also, luminal endoprostheses with an expandable coating on the surface of external walls of radially expandable tubular supports are proposed in U.S. Pat. No. 4,739,762 and U.S. Pat. No. 4,776,337. In these two patents, the coating is made from thin elastic polyurethane, Teflon film or a film of an inert biocompatible material. A. Balko et al., “Transfemoral Placement of Intraluminal Polyurethane Prosthesis for Abdominal Aortic Aneurysm”, Journal of Surgical Research, 40, 305-309, 1986, and U.S. Pat. No. 5,019,090 and U.S. Pat. No. 5,092,877 mention the possibility to coat stent materials with porous or textured surfaces for cellular ingrowth or with non-thrombogenic agents and/or drugs.

While the above methods have shown some promise with regard to treating certain aneurysms with non-invasive methods, there remains a need for an endovascular graft system which can be deployed percutaneously in a small diameter flexible catheter system. The present invention satisfies these and other needs.

It is a general object of the present invention to provide an improved luminal or endovascular graft that is expandable in place and, once expanded, can be rendered self-supporting.

Another object of this invention is to provide biocompatible endovascular grafts that are expandable in vivo and are supportive once so expanded.

Another object of the present invention is to provide an improved expandable reinforced graft that can be delivered by way of a balloon catheter or similar device, whether in tubular or bifurcated form.

Another object of this invention is to provide an improved endovascular graft which fully covers diseased or damaged areas for carrying out luminal repairs or treatments.

Another object of the present invention is to provide an improved endovascular graft wherein the endoprothesis is substantially enclosed within biocompatible elastomeric material which is presented to the surrounding tissue and blood or other body fluid.

Another object of this invention is to provide an expandable, supportive endovascular graft that can be tailored to meet a variety of needs, including a single graft designed to address more than a single objective.

### SUMMARY OF THE INVENTION

One embodiment of the invention relates to a coaxial double lumen tube adapted for forming an endovascular graft which comprises an outer tube positioned over an inner tube, both tubes being made of a material acceptable for use in endovascular grafts and having an internal and external diameter and a wall thickness, the outer tube having an internal diameter and the inner tube having an external diameter such that a space is created between the outer tube and inner tube, the space being at least partially filled with an uncured adhesive which, upon curing after endovascular implantation, cures to adhere the inner and outer tubes together to form a self-supportive endovascular graft.

A second embodiment of the invention concerns a method of deploying an endovascular graft adequate for maintaining a flow of blood therethrough and preventing leakage or failure of a compromised portion of a patient's body lumen within the patient's body lumen, comprising:
a) providing a coaxial double lumen tube adapted for forming an endovascular graft which comprises an outer tube positioned over an inner tube, both tubes being made of a material acceptable for use in endovascular grafts and having an internal and external diameter and a wall thickness, the outer tube having an internal diameter and the inner tube having an external diameter such that a space is created between the outer tube and inner tube, the space being at least partially filled with an uncured adhesive which, upon curing after endovascular implantation, cures to adhere the inner and outer tubes together to form a self-supportive endovascular graft,
b) percutaneously delivering the double lumen tube through a delivery catheter system to the compromised portion of the patient's body lumen; and
c) following positioning of the double lumen tube within the patient's lumen, curing the adhesive to adhere the inner and outer tubes together to form a self-supportive endovascular graft.

Still another embodiment of the invention comprises a kit with components suitable for forming an endovascular graft adequate for maintaining a flow of blood therethrough and preventing leakage or failure of a compromised portion of a patient's body lumen comprising two tubes configured to be an outer tube positioned over an inner tube, both tubes being made of a material acceptable for use in endovascular grafts and having an internal and external diameter and a wall thickness, the outer tube having an internal diameter and the inner tube having an external diameter such that a space is created between the outer tube and inner tube when so positioned, and an uncured adhesive for at least partially filling the space when created by the positioning, the adhesive, upon curing after endovascular implantation, cures to adhere the inner and outer tubes together to form a self-supportive endovascular graft.

A still further embodiment of the invention concerns an article of manufacture comprising packaging material and the coaxial double lumen tube described above contained within the packaging material, wherein the coaxial double lumen tube is effective for implantation in a patient, and wherein the packaging material comprises a label which indicates that the coaxial double lumen tube can be used for such implantation.

### BRIEF DESCRIPTION OF THE DRAWINGS

FIGS. 1a-1j depict various views of the endoluminal structure of the invention during construction.

FIGS. 2a-2c depict various views of an endoluminal structure of a specific design.

FIG. 3 is a schematic diagram of another specific endoluminal structure.

FIGS. 4a-4d are schematic diagrams of the flow system employed to test an endoluminal structure.

FIGS. 5-7 depict angiographies demonstrating results of tests employed utilizing the endoluminal structures of the invention.

### DETAILED DESCRIPTION OF THE INVENTION

This invention generally relates to supportive endoluminal grafts which have the ability to be delivered transluminally and expanded in place to provide a graft that is endoluminally positioned and placed, with the aid of an appropriate catheter, and that remains so placed in order to both repair a vessel defect and provide lasting support at the location of the graft. More particularly, the graft combines into a single structure both an expandable luminal prosthesis tubular support component and an elastomeric graft component wherein the material of the graft substantially covers either the internal, the external or both of the internal and external surfaces of the expandable tubular support component. When desired, the expandable supportive luminal graft takes on a bifurcated structure for repair and support of vessel locations at or near branching sites. The graft component is stretchable or elastomeric and does not substantially inhibit expansion of the tubular support component while simultaneously exhibiting porosity which facilitates normal cellular growth or invasion thereinto of tissue from the body passageway after implantation.

By the present invention, grafts which are expandable and supportive are provided that expand from a first diameter to a second diameter which is greater than the first. When it is at its first diameter, the expandable supportive graft is of a size and shape suitable for insertion into the desired body passageway. The material of the graft is substantially inert and has a generally cylindrical cover and/or lining generally over the outside and/or inside surface of the expandable supportive component. The cover and/or lining is especially advantageous because it is elastomeric and porous to encourage desirable growth of tissue thereinto in order to assist in non-rejecting securement into place and avoidance of stenosis development. When a bifurcated expandable supportive luminal graft is desired, the porous, elastomeric liner and/or cover is secured over a bifurcated expandable understructure. The material must be elastomeric enough to allow for expansion by up to about 2 to 4 times or more of its unexpanded diameter.

The present invention is directed generally to a system and method for treatment of a body lumen or passageway within a patient's body. More specifically, the invention is directed to an endovascular graft for treatment of weakened or diseased blood vessels. The system of the present invention may delivered intraoperatively, but is preferably delivered percutaneously.

The endoluminal structure of the invention can be considered as a second-generation microstent graft because of the following features.

Novel design: Unlike the metallic stents, the device of the invention is made from highly flexible elastomers. The hardening material is a low viscous liquid adhesive which will have negligible impact on the overall device flexibility. Balloon catheters, which are designed for intracranial application, are preferably used for the present application. Moreover, the increased flexibility will also allow the micrograft placement for previously inaccessible lesions, in other areas of the human body.

Reconstrainable/repositionable: The micrograft device can be balloon inflated and deflated multiple times to obtain optimal positioning before permanent deployment.

Detachable: The micrograft will only be detached, once placement is ideal, as determined by high quality imaging, by triggering the polymerization reaction. This feature will reduce the risk of unsatisfactory deployment. Currently, there are no stents, covered stents, or stent-grafts that are retrievable once deployed; if the position is unsatisfactory, nothing further can be done, other than to place another second permanent stent.

Numerous applications: This micrograft of the invention can be applied to an enormous array of luminal pathologies, and will be used as an alternative to traditional metallic and polymer covered metallic stents for treatment of urinary, biliary, bronchial, aortic, coronary, intracranial and peripheral vascular diseases.

Ultrasound compatible: In clinical practice, intravascular ultrasound (IVUS) is most often used as an adjunct to balloon angioplasty to detect dissection, stent underdeployment, stent thrombosis and to predict restenosis risk. It is also used as an accessory to diagnostic angiography to evaluate lesions of uncertain severity and to detect disease, which is not visible on angiography. The polymeric design of the micrograft will allow improved co-axial intravascular ultrasound imaging for improved graft placement.

Generally, the coaxial double lumen tube of the invention may be constructed and utilized as follows. In the first step, a double lumen cylindrical tube is made from two different size seamless tubes. In the second step, the space between the two lumens is filled with a, e.g., ultraviolet curable adhesive. To clearly visualize the device before and after the deployment under a fluoroscopic guidance, either the tube material or the adhesive is preferably blended with a radio-opaque material. The third step is the installation of the device on a balloon catheter. The final step is the deployment of the device followed by the fast curing of the adhesive material. The tube remains self-supporting in its expanded form when the balloon is deflated and taken out. Specific details of the method of making the double lumen tube are set forth in the non-limiting examples below.

### EXAMPLE 1

### Method of Making Uniform Polymer Tubes

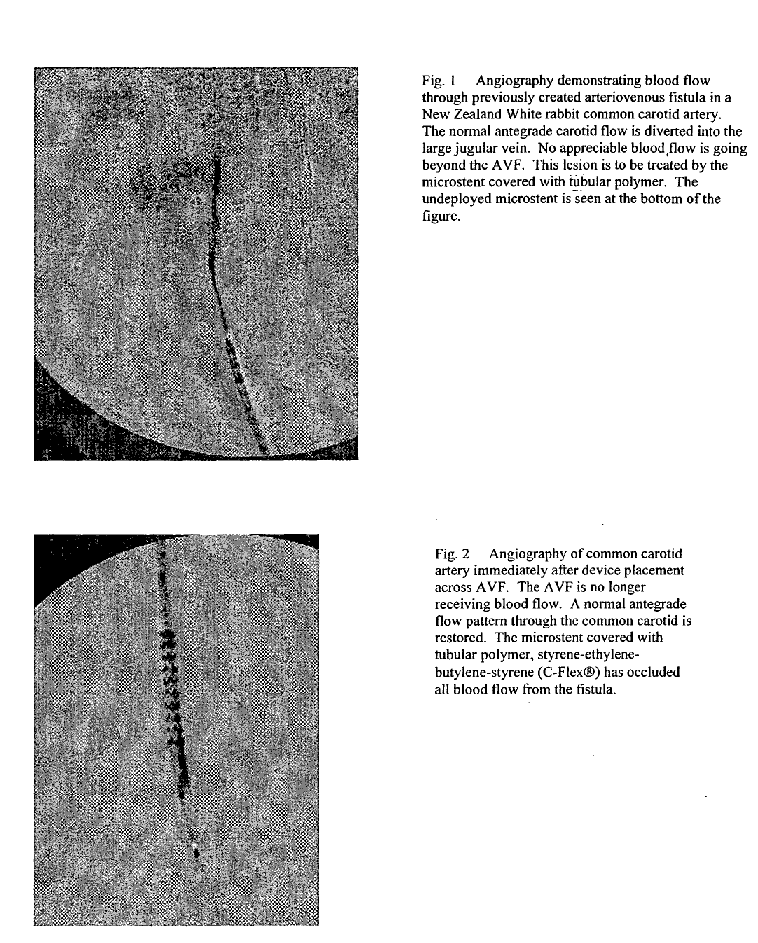

Polymer tubes were made from Carbosil 40 90A (The Polymer Technology Group Inc., Berkeley, Calif., a solution grade elastomeric, tear-resistant silicone-polyurethane thermoplastic copolymer) by the dip-coating method. Two stainless steel rods of appropriate sizes were used as mold substrates (cores). Each rod is 1.5 inches long and the outer diameter (OD) of the rod represents approximately the inner diameter (ID) of the resulting tube. Each rod was dipped twice in 15 wt % polymer solution in tetrahydrofuran (THF) at about a 4 mm/sec withdrawal rate and a 30 minute interval between two successive dips to form an approximately 150 μm thick polymer coating. Coated rods were then vacuum-dried. Coating on both ends of the rod was non-uniform. Therefore, keeping a uniform section of 20 mm long coating around the middle of the rod, the remaining coating was carefully cut by a sharp surgical razor blade and then removed. The coated rod was then immersed in 70% acetone solution for 10-15 minutes to swell the polymer coating. Finally the coating was detached from the rod in the form of a tube. Tubes of two different sizes (ODs are 2.0 mm for the inner tube and 3.2 mm for the outer tube) were made as shown in FIG. 1a.

### EXAMPLE 2

### Method of Making the Double Lumen Tubular Graft

As shown in FIG. 1b, two polymer rims were made at both ends of the inner tube. To make the rim, a drop of polymer solution (Carbosil 40 90A in THF) was smeared around the tube uniformly and then allowed to dry. The purpose of making the polymer rim is that once it is placed inside the outer tube it will not move and, also the sealing of the two tubes at the rim location is facilitated. In the next step, the inner tube was inserted completely inside the outer tube (FIG. 1c). Two elastomeric tubular connectors (Silastic, Dow Corning, 300 μm ID and 630 μm OD) were then introduced in between the inner and outer tubes to get access to the cylindrical pocket between the tubes (FIG. 1c). One connector is for the adhesive injection (inlet) and another is for the trapped air removal (outlet). Next a stainless steel rod of appropriate size was placed inside the inner tube and then both ends of the two tubes were completely sealed at the rim location with a few drops of Carbosil 40 90A solution (FIG. 1d). Polymer rims prevented the penetration of polymer solution inside the cylindrical pocket and the stainless steel rod kept the shape of the inner tube intact, keeping it from collapsing during the sealing process. Once dried, the rod was removed. The double lumen tubular graft, thus formed, was then tested to be sure that there was no leakage. The entire procedure was performed under a microscope.

### EXAMPLE 3

### 3.1.3 Adhesive Injection

The double lumen tubular graft was then wrapped with aluminum foil to protect it from light and placed vertically, keeping both ports facing upward. Light curable liquid adhesive (Loctite 4304 light cure adhesive, LOCTITE, Rocky Hill, Conn.; specific gravity 1.07 and viscosity 20 cP at 25° C.) was then injected through the inlet and the trapped air was removed through the outlet by applying a mild suction. Once the cylindrical pocket was completely filled with the adhesive, both ports were cut, removed and the holes sealed with Carbosil 40 90A solution. The flexibility of the graft filled with the adhesive did not change, as shown in FIG. 1e.

### EXAMPLE 4

### 3.1.4 Installation of the Micrograft Device on a Balloon Catheter

The micrograft device was then installed onto a 20 mm long, 5.5 mm nylon balloon of a percutaneous transluminal angioplasty (PTA) dilatation catheter (Cordis Corporation, Miami, Fla.; FIG. 1f). To secure the graft, the ID of the micrograft was made slightly smaller than the balloon diameter in its constricted form. The balloon was wiped with lubricant (vegetable oil) for easy insertion of the balloon catheter inside the tubular graft.

### EXAMPLE 5

### Deployment of the Micrograft Device

Using a digital inflation device and fluid dispensing syringe, saline pressure was applied to the balloon. FIG. 1g and 1h show the micrograft size at the balloon inflation pressure of 2.0 and 10.0 atm respectively. Keeping the balloon in the inflated condition, the micrograft was exposed to a handheld UV light source (Spectroline®, model; ENF-240C) for 2 minutes. The graft was rotated by hand along the catheter axis to make sure that the adhesive was exposed to the UV light thoroughly. The balloon was then deflated and removed from the graft. The graft was self-supportive, holding its expanded form intact as shown in FIG. 1i. A cross-section of the deployed graft (4.8 mm ID and 5.9 mm OD) is compared with a cross-section of its tubular components in FIG. 1j.

Typical components and parameters of the system for manufacturing, assembling and delivering the tube for use as an endovascular graft include:

Materials for making tubes: Tear-resistant, biocompatible elastomeric polymer materials are preferably employed for making tubes. Suitable materials include polyurethane (Tecoflex® SG-80A, Thermedics Polymer Products, Woburn, Mass.) and silicone-polyurethane copolymers (Carbosil 40 90A, PurSil 20 80A and PurSil AL5 75A, The Polymer Technology Group Inc., Berkeley, Calif.). Tecoflex® SG-80A is an aliphatic polyether based thermoplastic polyurethane (TPU). PurSil™ silicone-polyether-urethane and CarboSil™ silicone-polycarbonate-urethane are copolymers containing silicone in the soft segment. PurSil 20 80A is an aromatic silicone polyetherurethane whereas PurSil AL5 75A is an aliphatic silicone polyetherurethane. The following table lists some of the physical test data of these materials reported by the manufacturers. From the table it is clear that these materials cover a range of mechanical properties. The optimal material for each application can be selected based on a study of these and similar properties.

| | Tensile | Tensile | | | |
|---|---|---|---|---|---|
| | Stress at | Stress at | Ultimate | | Tear |
| | 100% | 300% | Tensile | Ultimate | Strength, |
| | Elongation | Elongation | Strength | Elongation | die “C” |
| Elastomer | (psi) | (psi) | (psi) | (%) | (psi) |
| Tecoflex ® | 300 | 800 | 5800 | 660 | N/A |
| EG-80A* | | | | | |
| PurSil ™ | 270 | 570 | 5300 | 900 | 390 |
| 20 80A | | | | | |
| PurSil ™ | 900 | 1630 | 4900 | 770 | 115 |
| AL5 75A | | | | | |
| CarboSil ™ | 1310 | 2400 | 4300 | 530 | 500 |
| 40 90A | | | | | |

*Represents the test data for extrusion grade, the solution grade data are not available. The solution grades differ from the extrusion grades in that they contain no melt processing lubricants.

If necessary, in order to clearly visualize the micrograft under a fluoroscope while performing in vivo tests, the tubes can be made radiopaque. Radiopaque grade Tecoflex® is available with 20 wt % and 40 wt % loading of barium sulfate. Other polymers can also be blended with barium sulfate or other radiopaque substance.

Method of making tubes: A dip-coating method is preferably used to make the polymer tubes. Tecoflex® EG-80A is soluble in N,N-dimethylacetamide (DMAC) and other silicone-polyurethane copolymers are soluble in tetrahydrofuran (THF). Tubes will be made using stainless steel rods of various sizes (minimum diameter about 1.0 mm and maximum diameter about 5 mm) as mold substrate. Tube thickness depends on three parameters, the polymer concentration, the total number of dips and the withdrawal rate. By proper adjustment of these three parameters tubes of about 150 μm thickness can be made.

Method of making double lumen coaxial micrograft: As described above, the tubular components are assembled together to make a micrograft. Rotational molding offers design advantages over other molding processes. With proper design, parts that are assembled from several pieces can be molded as one part, eliminating expensive fabrication costs. The process also has a number of inherent design strengths, such as consistent wall thickness.

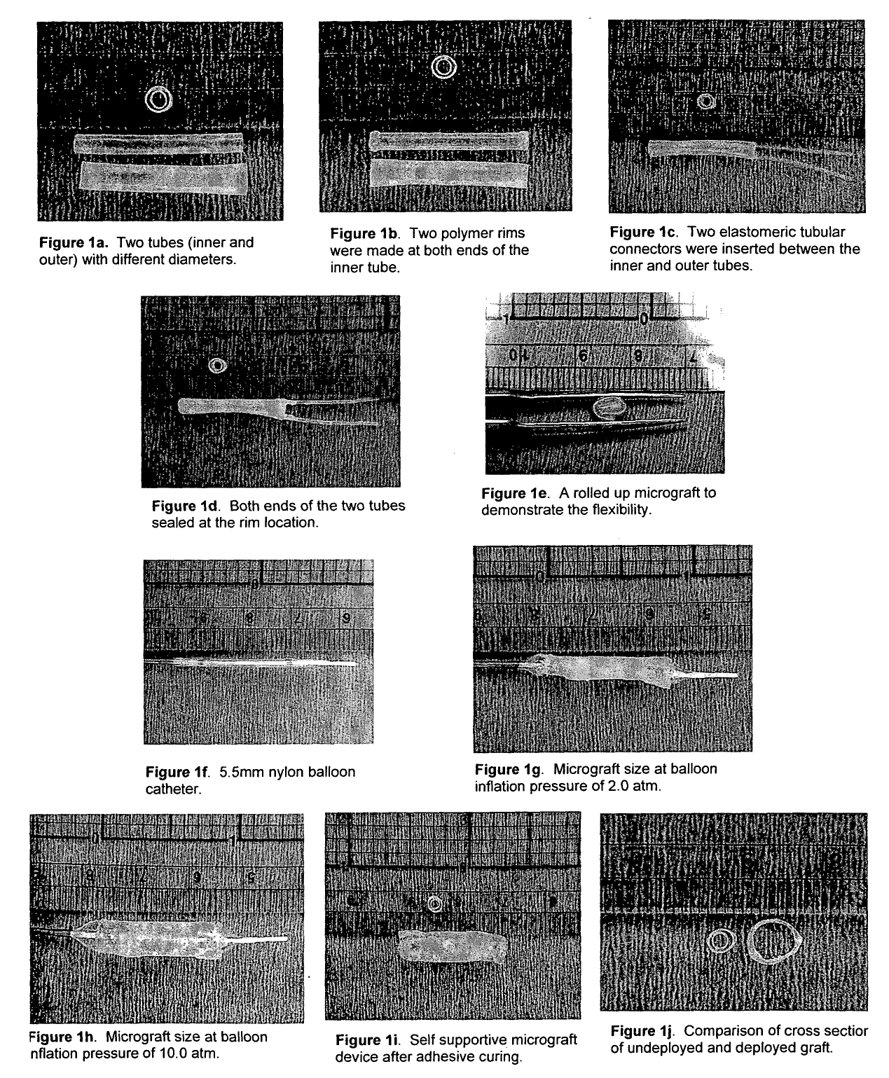

Special micrograft design: In some instances, the placement of the micrograft device may block the blood flow of a nearby perforating artery. To address this problem a specially designed device with a hole as shown in FIG. 2a can be utilized. Its longitudinal cross section will appear as shown in FIG. 2b. FIG. 2c shows the schematic representation of the location of the device when it is deployed across a wide necked aneurysm. It has been reported that placing an implant device in arteries less than 4 mm diameter (4 mm barrier) can cause thrombosis. Having multiple holes that would allow endothelial cell growth and reduce the risk of thrombosis could solve this problem. Another possible way to reduce this risk is to coat the graft with anti-thrombogenic material such as heparin.

Liquid adhesive injection: Once the double lumen micrograft is made, as described, liquid adhesive is injected into the cylindrical pocket followed by the sealing of both the inlet and outlet ports. If required, liquid adhesive will be blended with micronized tantalum powder in order to provide contrast for fluoroscopy. The pressure injection method will also be used for efficient adhesive injection. The device will then be thoroughly inspected under a microscope to be sure that there is no leakage. Care will be taken to avoid the exposure of the micrograft to light because this might trigger the polymerization reaction of the liquid adhesive materials. The strength of the graft will depend on the amount of the adhesive material. Obviously, the greater the amount of adhesive material the greater will be the strength.

Adhesive triggering mechanism: The liquid adhesive blend solidifies when the monomer component in the adhesive is polymerized. This initiation (triggering) can be carried out in two ways, internal and external.

External triggering mechanism (triggering by light): In order for a light cure adhesive to react to UV or visible light, a chemical called a photoinitiator (catalyst) must be present in the formulation. Light emitted from a suitable source causes the photoinitiator to fragment into reactive species. These fragments initiate a rapid polymerization process with monomers and oligomers in the system to form a crosslinked, durable polymer. In other words, photoinitiators in the UV light curable cyanoacrylate absorb light energy and dissociate to form radicals that trigger the polymerization process in the adhesive. In actual practice the UV light can be transported through an optical fiber, which will be navigated through the balloon port of the catheter. The cladding will be removed from the tip of the fiber-optic to get exposure to UV light.

Internal triggering mechanism (triggering by anions): Termed pressure-sensitive triggering, polymer microspheres of sponge materials (e.g. PVA microspheres, ˜700 microns) are used to trap alkaline water (pH: ˜8.5) or a mixture of water and dimethylsulfoxide (DMSO, to aid the mixing process of cyanoacrylate monomer and the catalyst water for anionic polymerization) solvent in their spongy compartments followed by a thin coating with a non-porous brittle polymer materials (e.g. PMMA, polystyrene). These microspheres function as a catalyst for the polymerization reaction of the alkylcyanoacrylate monomer. The double lumen compartment will be filled with monomer, pre-polymers and microsphere-catalyst. Upon deployment of the balloon, the catalyst (anions) will be squeezed out of the microspheres by external deployment pressure and will trigger the polymerization reaction. In general, cranioplastic cement is used to repair defects in the skull, together with the titanium mesh. Cranioplastic cement is generally available in powder form, a blend of methylmethacrylate polymer, methylmethacrylate-styrene copolymer (99.0%-99.6%, W/W), benzoyl peroxide (0.4%-1.0%, W/W), and liquid (methylmethacrylate monomer). The cement mixture requires about an hour to harden. Both cyanoacrylate and methylmethacrylate are vinyl monomers and the polymerization reaction mechanisms are similar. Replacing liquid methylmethacrylate monomer with ethylcyanoacrylate leads to a fast polymerization reaction. A benzoyl peroxide triggered polymerization reaction of ethylcyanoacrylate occurs in seconds.

The following tests can be performed to evaluate the robustness of the device fabrication and to determine the device performance in vitro.

### EXAMPLE 6

### Mechanical Testing of the Device by Instron

Mechanical testing of the micrograft device is performed using an Instron model 4301 (Instron Corporation, Canton, Mass.). Using an appropriate load cell, a complete stress-strain profile for both tubings is generated. Since the mechanical properties of the micrograft are changed if it is exposed to the light mechanical testing is preferably performed in the dark. Using the Instron, the strength of the device in its expanded form (after curing) is also evaluated by mechanical testing. The force required to deform the device (plastic deformation) will be correlated to the amount of injected adhesive.

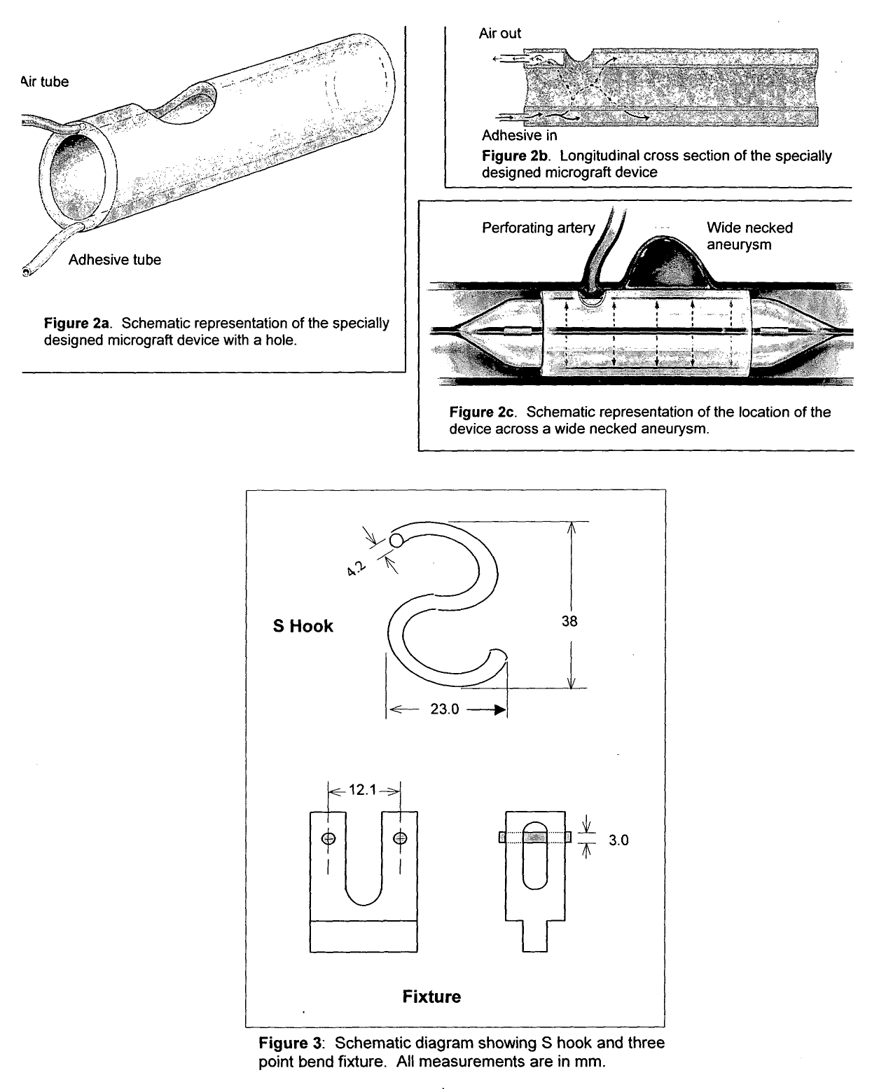

Flexibility and maneuverability testing are necessary to predict the feasibility of navigating the device through the tortuous intracranial vasculature system in the brain before the final deployment. A three-point bend test is performed on the double lumen tubular micrograft after the adhesive injection. As shown in FIG. 3, a set up (a bend test fixture and an “S” hook) for the bend test is designed and manufactured. The micrograft tends to kink while performing the bend test. To overcome this problem, compliant (highly flexible) silicone rods of appropriate size are made and used as substrate for the tube. Once the tube is mounted over the rod it will not kink while performing the bend test. Silicone rods are made from two-component platinum cure Silastic® T2 (Dow Corning, Midland, Mich.) mold making rubber. A melting point glass capillary tube of suitable size (Kimax-51 borosilicate glass from Kimble-Kontes, Vineland, New Jersey) is used as a mold substrate. Once cured inside the capillary tube, the silicone rods are removed from the mold by dissolving the glass in hydrofluoric acid. This experiment can be used to test the device both in vitro and in vivo.

### EXAMPLE 7

### Performance Testing of the Device in an In Vitro Flow System

An in vitro flow system is used for device performance testing. The flow system approximately mimics the blood circulation in the brain. A simulated blood fluid (SBF) is circulated through the flow system in a pulsatile manner. Three models are employed: a human middle carotid artery model (MCA) for flexibility and maneuverability testing, an aneurysm model and an AVF model for the device in vitro performance testing. The feedback from in vitro testing helps further the development of the design and fabrication process.

The following materials are used in the in vitro flow system: Masterflex® variable speed peristaltic pump (Cole Parmer, Niles, Ill.), Tygon® tubing (Fisher Scientific, Fairlawn, N.J.), quick disconnect fittings (Fisher Scientific, Norcross, Ga.), polyethylene and polypropylene tubings (Clay Adams, Parsippany, N.J.), sheath introducer (Cordis Endovascular Systems, Miami, Fla.), a 2-way PTFE plug stopcock (Kimax, Kimble-Kontes, Vineland, N.J.) as flow resistor, a Shimpo digital force gauge, model FGV (A)-5A, capacity 5.0 lb (Davis Inotek Instruments, Baltimore, Md.) and a closed reservoir. The simulated blood fluid (SBF) is used for flow experiments. The SBF is comprised of the following materials: poly(vinyl alcohol) (PVA) with a molecular weight of 93,400 (Eastman Kodak, Rochester, N.Y.), sodium chloride (NaCI) (Fisher Scientific, Fairlawn, N.J.), boric acid (Sigma Chemicals, St. Louis, Mo.), and sodium tetraborate decahydrate (Aldrich Chemical Company, Inc., Milwaukee, Wis.).

The following materials and equipment are used for the data acquisition component of the flow system: a desktop computer, a multifunction I/O data acquisition board (Model PC-LPM-16/PnP) (National Instruments®, Austin, Tex.), NI-DAQ software Version 6.7 (National Instruments®, Austin, Tex.), LabVIEW™ 5.1 software (National Instruments®, Austin, Tex.), an Archer breadboard (Radio Shack®, Fort Worth, Tex.), a 50-pin ribbon cable, silicon pressure sensors with a range of 0 to 7.3 psi (MPX5050 series, Motorola, Phoenix, Ariz.), and a flow sensor with a range of 60mL/min to 1,000 mL/min (Model 101T, McMillan Company, Georgetown, Tex.). The SBF was made using a procedure from Jungreis and Kerber [“A solution that simulates whole blood in a model of the cerebral circulation.” Am J Neuroradiol. 12(2): 329-330 (1991)]. First, 12.1 g of PVA are dissolved in one liter deionized water. In a separate container, 23.2g of sodium borate are dissolved in deionized water. The two solutions are mixed and diluted to three liters. Boric acid is then added to lower the pH to 7.5.

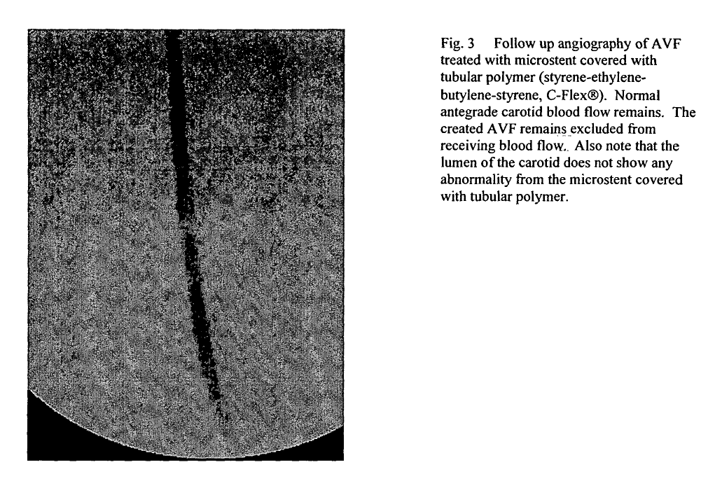

A schematic of the in vitro flow system is shown in FIG. 4. The flow system consists of two components, an electronic component and the flow component. The electronic component includes a breadboard and a computer with DAQ board. The computer is connected to the breadboard as shown in FIG. 4a. The flow component includes a peristaltic pump, a catheter introducer, a model of interest, a resistor, a flow meter, a closed reservoir (not shown in the schematic drawing) and several pressure sensors; all connected in series. The device performance is studied in three different models, a middle carotid artery (MCA, FIG. 4b) model, an aneurysm model (FIG. 4c) and an arteriovenous fistula model (AVF, FIG. 4d). The pressure sensors (P1, P2, P3, P4, and P5) monitor the SBF pressure at different locations (as shown in FIG. 4b, 4c and 4d) and the flow meter monitors the SBF flow in the flow system. All sensors (pressure and flow) are connected to the breadboard. The resistor will model normal brain capillary bed. The micrograft device is introduced through the catheter introducer port. The pulsatile flow rate is controlled by the peristaltic pump.

Middle Carotid Artery (MCA) model for the flexibility and maneuverability testing: A polypropylene tube of 2.5 mm ID is used for making the model of the tortuous MCA. This type of tube may kink while bending it to give the tortuous shape. To overcome this problem, an appropriate size copper wire is inserted first inside the tube to give the right shape. Translucent silicone adhesive (Silastic T2 from Dow Corning) is applied over the tube and then it is heat-treated. Once cured, silicone oil is injected into the tube to make the tube interior slippery. Then the structure is straightened and the copper wire support removed. Once released, the tube will return to its tortuous shape. The silicone over coating should reinforce and retain the structure. It is then be cleaned and installed as shown in FIG. 4b. The MCA model is connected to the flow system to test the flexibility and maneuverability of the micrograft device at different locations (A, B, C, D and E) as shown in FIG. 4b. The force required to push the catheter through the MCA model is measured by a digital force gauge and then a histogram showing force at different locations is created.

Aneurysm and AVF model: As shown schematically, both aneurysm (FIG. 4c) and AVF (FIG. 4d) models are made from silicone polyurethane copolymers (e.g. Carbosil 40 90A) by using appropriate molds. As described before the dip-coating method is used to make tubular components for AVF and tubular and balloon components for the aneurysm model. The interior of the balloon and the tubes are coated with a hydrophilic coating (Hydromed C™, CT biomaterials, CardioTech International, Inc., Woburn, Mass.). For in vitro performance testing, these models are then connected to the flow system.

### EXAMPLE 8

Safety and efficacy testing of the device in an in vivo rabbit model. In vivo arteriovenous fistula creation (AVF): Under general anesthesia, New Zealand White (NZW) rabbits are placed in dorsal recumbency and the ventral cervical region shaved and prepared for surgery. At this time, 100 IU/kg of heparin is administered via the marginal ear vein. A longitudinal ventral midline incision is made above the right common carotid artery (CCA). The artery is isolated from the carotid sheath and right external jugular vein (EJV) dissected free of surrounding tissues. Flow through the CCA will be interrupted with proximal and distal atraumatic microvascular clamps while the EJV will be clamped proximally. The EJV is transected distally after the most distal part of the exposed vein is tied off. A 5-mm slit is made in the lateral wall of the CCA. An end to side anastomosis is created between the vein and artery using 10-0 polypropylene suture under an operating microscope. Flow is then re-established through the fistula by releasing the carotid artery clamps, followed by the EJV clamp. The incision is closed using cuticular PDS sutures and tissue adhesive. The animal is recovered and allowed to heal for 16 days prior to the next procedure.

### EXAMPLE 9

### In Vivo Aneurysm Creation

Under general anesthesia, NZW rabbits are placed in dorsal recumbency and the ventral cervical region shaved and prepared for surgery. A longitudinal ventral midline incision is made above the right common carotid artery (CCA). The artery is isolated from the carotid sheath and dissected free of surrounding tissues. Two lengths of suture material are placed around the CCA. One of the sutures is used to ligate the CCA distally. The other suture is placed around the CCA 1-2 cm proximal to the first suture and will provide control of blood flow via traction during sheath placement. Once this is accomplished a small slit is made in the artery and a vascular sheath is placed. Once the sheath is in place, the second suture is used to secure it in place. An endovascular balloon catheter is then inserted through the sheath and advanced to the CCA origin. The balloon is inflated to create an isolated space inside the artery. Porcine pancreatic elastase is then injected through the sheath into the arterial space while the balloon catheter is still in place. The elastase is allowed to incubate inside the arterial lumen for approximately 20 minutes. After this incubation period, the elastase is aspirated out of the vessel lumen, the balloon is deflated, and the catheter removed. The sheath is also removed and the suture material tightened to ligate the artery. The incision is then closed and the animal recovered.

Three weeks after the first procedure, the animal is again put under general anesthesia and an IV catheter is placed in the cephalic vein. Magnetic resonance angiography (MRA) is used to determine the presence, size and shape of the created aneurysms. Immediately prior to starting MRA, 2 ml Gadolinium contrast media is administered intravenously through the catheter. After MRA is performed, the IV catheter is removed and the rabbit is recovered.

In vivo testing of the micrograft device: After the designated wait period, a second procedure is performed to cure the vascular lesion (aneurysm or AVF) with the polymeric endovascular micrograft. Before induction of anesthesia, a combination of aspirin and Plavix (10 mg/kg of each) is administered by mouth. The animal is placed under general anesthesia and the medial aspect of the right hind limb shaved and prepared for surgery. A small skin incision is made to expose the femoral artery for sheath placement. The artery is ligated distally and a vascular sheath is introduced into the femoral artery. Before introduction of the catheter supporting the micrograft, heparin (100 IU/kg) is administered IV. Under fluoroscopic guidance, the catheter supporting the micrograft is advanced through the vasculature to the site of the lesion. Once properly placed, the micrograft is deployed. The results of the micrograft placement is observed using digital subtraction angiography (DSA). The catheter and sheath is removed and the femoral artery ligated. The wound is closed and the animals is recovered and monitored for a period of two, four or six weeks, depending on the survival group designated. During this period, aspirin and Plavix (10 mg/kg PO) is administered daily.

### EXAMPLE 10

### Efficacy and Histological Compatibility Test

Under protocols approved by University of Florida's Institutional Animal Care and Use Committee, the invention was tested for efficacy and histological compatibility in New Zealand White (NZW) rabbits. The procedures are described below.

Histological analysis of the effect of device placement was conducted in the normal common carotid artery. A vascular sheath was placed in the femoral artery of a NZW rabbit. Using endovascular techniques, a microstent covered with tubular polymer was navigated through the vasculature towards the common carotid artery. The device was deployed within the vessel and angiography was performed to confirm patency. Following device placement, the animal was monitored for a period of two to six weeks depending on the experimental group to which it was assigned. At the end of the determined monitoring period, angiography of the stented vessel was performed to reveal angiographic patency. The animals were then euthanized and the vessels harvested for histological examination.

Efficacy was determined using a NZW rabbit arteriovenous fistula (AVF) model. An end-to-side AVF was created using the external jugular vein and common carotid artery. The next procedure was conducted after a healing period of about two weeks. Endovascular access was obtained through a vascular sheath in the femoral artery. A microstent covered with tubular polymer was advanced towards the AVF and placed across the lesion under fluoroscopic guidance. Once proper placement was determined, the device was deployed, occluding the AVF from arterial blood flow. Angiography was used to determine successful occlusion of the lesion from flow. The animals were monitored for periods of three to six weeks. Endovascular access was again obtained and angiography was performed in the same manner to evaluate the result of AVF treatment with the device. Following the evaluative angiography, the animal was euthanized and the vessels and device were removed for histology.

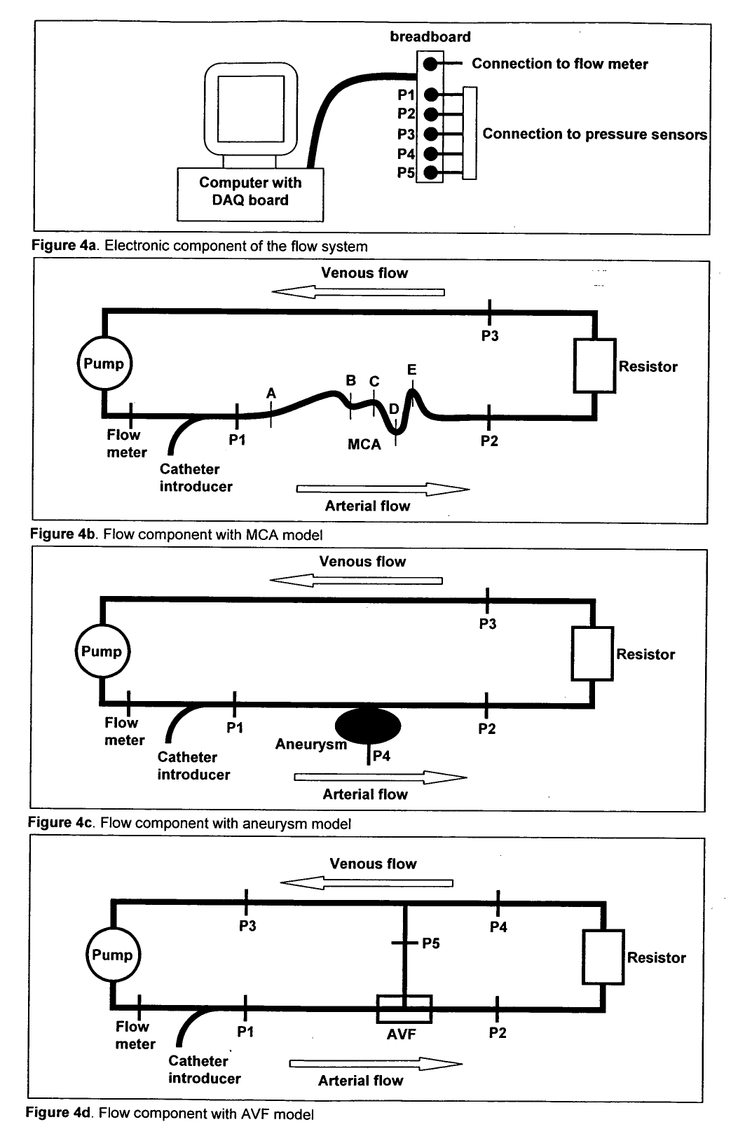

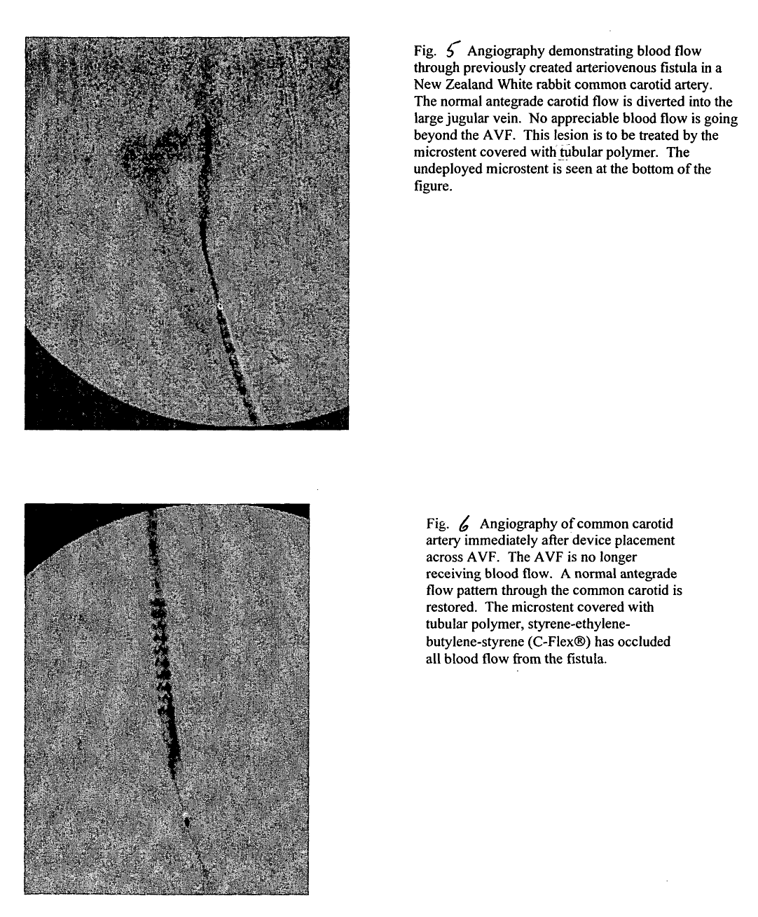

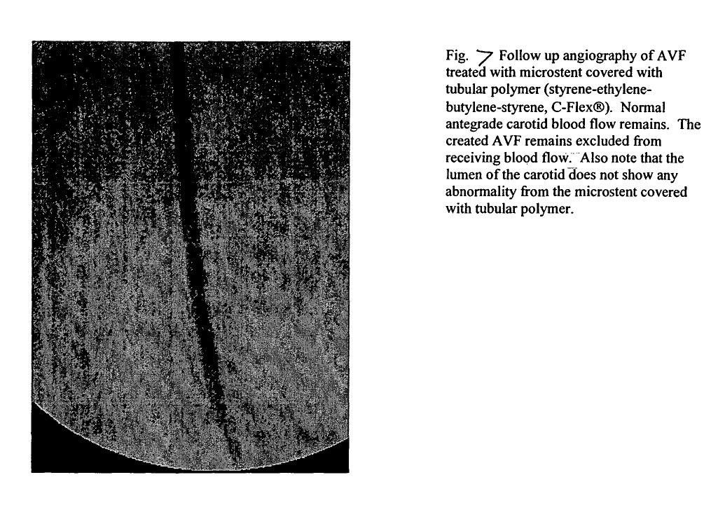

Various tubular polymeric materials were compared in these in vivo trials. Preliminary results are encouraging. The refined device was easily and reliably deployed. Placement of microstents covered with tubular polymers in normal carotid arteries resulted in minimal neointimal proliferation. In the AVF model, the device successfully occluded the lesion from flow while restoring normal flow through the primary vessel. After the monitoring periods, normal flow was still present and the lesions eliminated from circulation. Angiographies demonstrating the results of Example 10 are set forth in FIGS. 5-7.

It will be understood by those skilled in the art that the above constitutes a description of a preferred embodiments for manufacturing the device of the invention and that the invention is not limited thereto. For example, a variety of curing mechanisms may be employed to cure the adhesive employed between the tubes.

In the case of using light to cure the adhesive a photoinitiator or catalyst is usually employed to enable an efficient cure rate. Light emitted from a suitable source causes the photoinitiator to fragment into reactive species. These fragments initiate a rapid polymerization process with monomers and oligomers in the adhesive system to form a crosslinked, durable polymer. In other words, photoinitiators in the UV-light-curable cyanoacrylate absorb light energy and dissociate to form radicals that trigger the polymerization process in the adhesive. As described above in the preferred embodiment a UV curing system was employed. In general, electrophilic vinyl monomers such as cyanoacrylates are characterized by their high reactivity to anions such as OH_(hydroxyl) and NCS_(thiocyanates), and to Lewis bases such as amines and phosphines. Photo generation of thiocyanate, a known initiator, from Reinecke's salt (K⁺[Cr(NH₃)₂(NCS)₄]⁻, abbreviated to K+R−) in neat cyanoacrylate was found to lead to polymerization [Kutal C., Grutsch P A and Yang D B, Macromolecules, 24, 6872, 199 1 ]. Pt(acac)₂ (acac- is the anion of acetylacetone) may also used as a photoinitiator for the anionic polymerization of 2-cyanoacrylate (Lavallee R J, Palmer B J, Billing R, Hennig H, Ferraudi G, Kutal C, Inorganic Chemistr. 36, 5552, 1997). Many free radical initiators (such as benzoyl y peroxides) are also available for the polymerization of vinyl monomers such as acrylic types where polymerization stops as soon as the light is removed. Free radical acrylic systems are subject to oxygen inhibition, which means that oxygen in the air prevents the molecules at the surface from polymerizing, leaving a wet or tacky surface. It is desirable to polymerize the adhesive inside the graft materials in less than I minute and the glue formulation and light intensity are optimized to achieve this result. The UV light is transported to the in-vivo reaction site through optical fiber (UV compatible). The optical fiber is placed inside the balloon catheter. In order to cure uniformly the whole section of the graft, a required amount of cladding is stripped off at the distal end of the fiber so that a sufficient amount of UV light becomes available at the point of interest.

When employing mechanical triggering, instead of one there are two compartments inside the double lumen graft. One compartment will be filled with monomers and pre-polymers and the other compartment will contain catalyst (initiator). The separating membrane between these two compartments comprises a brittle material, e.g. PMMA, polystyrene etc. When inflated by the balloon, the brittle membrane separating two components breaks and allows the components to mix together. Thus, the polymerization reaction will start and the adhesive materials will be solidified.

In the case of pressure-sensitive triggering, polymer microspheres of sponge materials (e.g. PVA microspheres, ˜700 microns) can be used to trap alkaline water (pH: ˜8.5) or a mixture of water and dimethylsulfoxide (DMSO helps the mixing process of cyanoacryate monomer and the catalyst water for anionic polymerization) solvent in their spongy compartments followed by a thin coating with a non-porous brittle polymer materials (e.g. PMMA, polystyrene). These microspheres will behave as catalysts for the polymerization reaction of alkylcyanoacrylate monomer. The double lumen compartment is filled with monomer, pre-polymers and microsphere-catalyst. Upon deployment of the balloon, catalyst (anions) will be squeezed out of the microspheres by the external deployment pressure and will trigger the polymerization reaction.

Cranioplastic is a cement made with resins as the basic ingredients. It is used to repair defects in the skull in general together with a titanium mesh. It is generally supplied as a powder (a blend of methylmethacrylate polymer and methylmethacrylate-styrene copolymer (99.0% -99.6%, W/W) and benzoyl peroxide (0.4% -1.0%, W/W) and liquid (methylmethacrylate monomer) and must be mixed for its application. It takes about an hour to harden the materials. Both cyanoacrylate and methylmethacrylate are vinyl monomers and the polymerization reaction mechanisms are similar. Liquid methylmethacrylate monomer may be replaced with ethylcyanoacrylate. Benzoyl peroxide triggers the polymerization reaction of ethylcyanoacrylate and hardening occurs in seconds.

Electroactive polymers (EAP) have unique capabilities that enable new technologies (“Artificial Muscle”) and are susceptible to electrical triggering. Their attractive characteristics include the ability to induce large displacements and they may be employed to open a miniaturized valve system that will allow the mixing of catalyst with monomer in a more controlled way. Other possibilities include (i) electroosmotic transport of anions (catalyst) through a membrane separating the monomers and (ii) electrolytic dissolution (or pore formation) of ultrathin metallic membrane, which will allow mixing of the monomer and the catalyst.

The space between the tubes may be of any suitable size and shape. In the example above, the tubes were separated by about 0.425 mm. In addition, the graft may be made radio-opaque by incorporating heavy elements, which will show contrast in X-ray. Lipiodol, an iodinated poppy-seed oil is a good candidate, however, care must be taken that it not act to soften the graft. Tantalum powder and barium sulfate are commonly used radio-opaque materials, which can be mixed with the adhesive.

The tubes may be formed of any suitable polymeric material that is expandable, such as elastomers. The material must be biocompatible which means these materials will not be considered as foreign substances to the body immune system so that they will be suitable for implantation. There are many commercially available biocompatible materials, which are expandable. The ideal material will have low hardness, low modulus, high ultimate elongation, moderate-to-high tensile strength, high tear strength, abrasion resistance, excellent thromboresistance, biostability and long-term medical implant capability. It is also important that the hardening material (e.g. glue) does not interfere (e.g. swell, degrade and dissolve etc) with the tubing materials. The following elastomers have been found to be suitable.

The Polymer Technology Group (PTG), Inc., Berkeley, Calif. has introduced a series of interesting elastomeric polyurethane based biomaterials in their product line. ElasthaneTM polyetherurethane is a high-strength, aromatic thermoplastic with a chemical structure and properties very similar to Pellethaneo 2363 (Dow Chemical Company, Midland, Mich.) polyetherurethane series, which has been used to fabricate a large number of implantable devices, including pacemaker leads and cardiac prosthesis devices such as artificial hearts, heart valves, intraaortic balloons, and ventricular assist devices. Elasthane is designed for chronically implanted medical devices and demonstrates an impressive combination of mechanical properties and biological compatibility. Numerous medical devices and technologies have benefited from the combination of the exceptionally smooth surfaces, excellent mechanical properties, stability, and good biocompatibility of ElasthaneTM polyetherurethane. PTG recently received FDA (Food and Drug Administration) approval for the use of ElasthaneTm 55D and 75D thermoplastic polyetherurethane (TPU) in high- and low-voltage leads. They also have introduced a variety of silicone urethane copolymers. In this co-polymer series, PurSil-10 80A, PurSil-20 80A, PurSil AL-5 75A, CarboSil-40 90A are attractive candidates.

The thicknesses of the walls of the tubes may vary from about 50 to 1000 microns, depending upon the application desired.

Any suitable method of deploying the graft system of the invention may be employed. For example, the “intravascular ultrasound” method for aiding in the placement of the catheter may be employed. Intravascular ultrasound (IVUS) is an imaging modality in routine use in interventional coronary procedures. Several intraarterial ultrasound devices are commercially available. IVUS requires the threading of an ultrasound probe over a microwire through the area of interest. Cross sectional ultrasound pictures are then produced as the probe is slowly pulled back over the wire.

One of its most common uses has been in the determination of the adequacy of deployment of traditional metallic stents. The same method may be employed to situate the device of the invention. If the stent is in proper position, it can then be detached.

1-6. (Cancelled)

7. A coaxial double lumen tube adapted for forming an endolumenal graft, said double lumen tube comprising an outer tube positioned over an inner tube, both of said inner and outer tubes having an internal and external diameter and a wall thickness, said outer tube having an internal diameter and said inner tube having an external diameter such that a space is created between said outer tube and said inner tube, said space being at least partially filled with an uncured adhesive which, upon curing after endoluminal implantation, cures to adhere said inner and outer tubes together to form the endolumenal graft.

8. The coaxial double lumen tube of claim 7, wherein said endolumenal graft is selected from the group consisting of a vascular graft, a urinary graft, a biliary graft, and a bronchial graft.

9. The coaxial double lumen tube of claim 7, wherein said endolumenal graft is a vascular graft selected from the group consisting of an aortic graft, a coronary graft, an intracranial graft, and a peripheral vascular graft.

10. The coaxial double lumen tube of claim 7, wherein said double lumen tube has one or more bifurcations.

11. The coaxial double lumen tube of claim 7, wherein said double lumen tube has one or more holes perforating said inner tube and said outer tube.

12. The coaxial double lumen tube of claim 7, wherein said double lumen tube comprises a porous, flexible elastomer.

13. The coaxial double lumen tube of claim 7, wherein said double lumen tube comprises a polyurethane.

14. The coaxial double lumen tube of claim 7, wherein said double lumen tube comprises a silicone-polyurethane copolymer.

15. The coaxial double lumen tube of claim 7, wherein said double lumen tube comprises a polyurethane copolymer containing silicone in the soft segment.

16. The coaxial double lumen tube of claim 15, wherein said polyurethane copolymer is silicone-polyether-urethane or silicone-polycarbonate-urethane.

17. The coaxial double lumen tube of claim 7, wherein said double lumen tube comprises an aromatic silicone polyether-urethane or aliphatic silicone polyether-urethane.

18. The coaxial double lumen tube of claim 7, wherein said double lumen tube comprises a radio-opaque material.

19. The coaxial double lumen tube of claim 7, wherein said uncured adhesive comprises a radio-opaque material.

20. The coaxial lumen tube of claim 19, wherein said radio-opaque material is selected from the group comprising tantalum, barium sulfate, and lipiodol.

21. The coaxial double lumen tube of claim 7, wherein said uncured adhesive comprises a monomer that is polymerized upon triggering.

22. The coaxial double lumen tube of claim 21, wherein said polymerized monomer forms a cross-linked polymer.

23. The coaxial double lumen tube of claim 21, wherein said monomer is selected from the group consisting of methylmethacrylate, cyanoacrylate, and ethylcyanoacrylate.

24. The coaxial double lumen tube of claim 7, wherein said uncured adhesive comprises polymer microspheres and cyanoacrylate monomer.

25. The coaxial double lumen tube of claim 7, wherein said uncured adhesive is triggered to cure by pressure or electricity.

26. The coaxial double lumen tube of claim 7, wherein said uncured adhesive is curable by ultraviolet or visible light.

27. The coaxial double lumen tube of claim 26, wherein said uncured adhesive comprises cyanoacrylate.

28. The coaxial double lumen tube of claim 7, wherein said double lumen tube is imageable by ultrasound.

29. The coaxial double lumen tube of claim 7, wherein said double lumen tube is coated with an anti-thrombogenic material.

30. The coaxial double lumen tube of claim 29, wherein said anti-thrombogenic material comprises heparin.

31. The coaxial double lumen tube of claim 7, wherein said wall thickness of each of said inner and outer tubes is 50-1000 microns.

32. The coaxial double lien tube of claim 7, wherein said wall thickness of each of said inner and outer tubes is about 450 micrometers.

33. The coaxial double lumen tube of claim 7, wherein said space is about 0.425 mm.

34. The coaxial double lumen tube of claim 7, wherein said outerdiameter of said inner and outer tubes is 2.0 mm and 3.2 mm, respectively.

35. A system for treatment of luminal disorders, comprising the coaxial double lumen tube of claim 7 and a balloon catheter, wherein said balloon catheter is located within said endoluminal graft, and wherein said internal diameter of said inner tube is smaller than the diameter of said balloon in constricted form.

36. A method of deploying an endolumenal graft adequate for maintaining a flow of fluid therethrough and preventing leakage or failure of a portion of a patient's body lumen within the patient's body, comprising:
a) providing a coaxial double lumen tube adapted for forming an endolumenal grail which comprises an outer tube positioned over an inner tube, both tubes being made of a material acceptable for use in endolumenal grafts and having an internal and external diameter and a wall thickness, the outer tube having an internal diameter and the innertube having an external diameter such that a space is created between the outer tube and inner tube, the space being at least partially filled with an uncured adhesive which, upon curing after endolumenal delivery, cures to adhere the inner and outer tubes together to form a self-supportive endolumenal graft;
b) delivering said double lumen tube to the portion of the patient's body lumen; and
c) following delivering of said double lumen tube within said patient's lumen, curing said adhesive to adhere said inner and outer tubes together to form the self-supportive endolumenal graft.

37. The method of claim 36, wherein the patient's body lumen is selected from the group consisting of vascular, urinary, biliary, and bronchial.

38. The method of claim 36, wherein the patient's body lumen is a vascular lumen is selected from the group consisting of aortic, coronary, intracranial, and peripheral vascular.

39. The method of claim 36, wherein said delivering is carried out intraoperatively.

40. The method of claim 36, wherein said delivering is carried out percutancously.

41. The method of claim 36, wherein said delivering is carried out using ultrasound guidance.

42. The method of claim 36, wherein said delivering is carried out through a delivery catheter system.

43. The method of claim 36, wherein the uncured adhesive is triggered to cure by pressure or electricity.

44. The method of claim 36, wherein the uncured adhesive is triggered to cure by ultraviolet or invisible light.

45. The method of claim 44, wherein the uncured adhesive comprises a photoinitiator and a monomer, and wherein said curing comprises providing the uncured adhesive with ultraviolet light by an optical fiber which causes the photoinitiator to fragment and initiate polymerization of the monomer.

46. The method of claim 36, wherein the uncured adhesive comprises polymer microspheres and alkylcyanoacrylate monomer, and wherein said curing is triggered by external deployment pressure, which causes the polymer microspheres to release anions that trigger the polymerization of the alkylcyanoacrylate monomer.

47. The method of claim 36, wherein the patient's body lumen is a vascular lumen, wherein the double lumen tube comprises one or more holes, wherein the portion of the patient 's vascular lumen has a perforating artery, and wherein the double lumen tube is delivered such that the one or more holes are adjacent to the perforating artery, thereby permitting passage of blood flow through the perforating artery and the one or more holes.

48. The method of claim 36, wherein the portion of the body lumen has one or more branching sites, and wherein the endolumenal graft is bifurcated.

49. The method of claim 36, wherein said method further comprises repositioning the double lumen tube two or more times prior to said curing.

50. The method of claim 36, wherein said method further comprises inflating the double lumen tube two or more times prior to said curing.

51. A kit comprising components suitable for forming an endolumenal graft adequate for maintaining a flow of fluid therethrough and preventing leakage or failure of a compromised portion of a patient's body lumen, said endolumenal graft comprising two tubes configured to be an outer tube positioned over an inner tube, both tubes being made of a material acceptable for use in endolumenal grafts and having an internal and external diameter and a wall thickness, the outer tube having an internal diameter and the inner tube having an external diameter such that a space is created between the outer tube and inner tube when so positioned, and an uncured adhesive for at least partially filling said space when created by said positioning, said adhesive, upon curing after endolumenal implantation, cures to adhere said inner and outer tubes together to form a self-supportive endolumenal graft.

52. The kit of claim 51, wherein the tubes are configured to be deployed within a low profile delivery catheter system.

53. An article of manufacture comprising packaging material and the coaxial double lumen tube of claim 7 contained within said packaging material, wherein said coaxial double lumen tube is effective for implantation in a patient, and wherein said packaging material comprises a label which indicates that said coaxial double lumen tube can be used for such implantation.
